(19) United States
(12) Patent Application Publication
(10) Pub. No.: US 2011/0112608 A1
(43) Pub. Date: May 12, 2011

(54) Implant Power System

(75) Inventor: Clemens M. Zierhofer, Kundl (AT)

(73) Assignee: MED-EL ELEKTROMEDIZINISCHE GERAETE GMBH, Innsbruck (AT)

(21) Appl. No.: 12/942,728

(22) Filed: Nov. 9, 2010

Related U.S. Application Data
(60) Provisional application No. 61/259,854, filed on Nov. 10, 2009.

(51) Int. Cl. A61F 11/04

(52) U.S. Cl. 607/57

(57) ABSTRACT

A power supply system for an implant includes an external power supply module. The external power supply module includes a first power signal transmission module having a first coil for transmitting a first electrical power signal across the skin of a user to the implant, and a first rechargeable battery for supplying power to the power signal transmission module. The power supply system further includes an external charger for recharging the first rechargeable battery. The charger includes a second power signal transmission module having a second coil for transmitting a second electrical power signal to the first coil.

### CROSS-REFERENCE TO RELATED APPLICATIONS

The present application claims priority from U.S. provisional application Ser. No. 61/259,854, entitled “Implant Power Control,” filed Nov. 10, 2009, which is hereby incorporated herein by reference in its entirety.

### TECHNICAL FIELD

The present invention relates to implants, and more particularly, to a system and methodology to recharge a battery in an external power piece associated with an implant.

### BACKGROUND ART

Cochlear implants and other inner ear prostheses are one option to help profoundly deaf or severely hearing impaired persons. Unlike conventional hearing aids that just apply an amplified and modified sound signal; a cochlear implant is based on direct electrical stimulation of the acoustic nerve. Typically, a cochlear implant stimulates neural structures in the inner ear electrically in such a way that hearing impressions most similar to normal hearing is obtained.

More particularly, a normal ear transmits sounds as shown in FIG. 1 through the outer ear 101 to the tympanic membrane (eardrum) 102, which moves the bones of the middle ear 103 (malleus, incus, and stapes) that vibrate the oval window and round window openings of the cochlea 104. The cochlea 104 is a long narrow duct wound spirally about its axis for approximately two and a half turns. It includes an upper channel known as the scala vestibuli and a lower channel known as the scala tympani, which are connected by the cochlear duct. The cochlea 104 forms an upright spiraling cone with a center called the modiolar where the spiral ganglion cells of the acoustic nerve 113 reside. In response to received sounds transmitted by the middle ear 103, the fluid-filled cochlea 104 functions as a transducer to generate electric pulses which are transmitted to the cochlear nerve 113, and ultimately to the brain.

Some persons have partial or full loss of normal sensorineural hearing. Cochlear implant systems have been developed to overcome this by directly stimulating the user's cochlea 104. One type of cochlear prosthesis may include two parts: a speech processor 111 and the implanted stimulator 108. The speech processor 111 typically includes a power supply (batteries) for the overall system, a microphone, and a processor that is used to perform signal processing of the acoustic signal to extract the stimulation parameters. The speech processor may be a behind-the-ear (BTE-) device.

The stimulator 108 generates the stimulation patterns (based on the extracted audio information) that are sent through an electrode lead 109 to an implanted electrode array 110. Typically, this electrode array 110 includes multiple electrodes on its surface that provide selective stimulation of the cochlea 104. For example, each electrode of the cochlear implant is often stimulated with signals within an assigned frequency band based on the organization of the inner ear. The placement of each electrode within the cochlea is typically based on its assigned frequency band, with electrodes closer to the base of the cochlea generally corresponding to higher frequency bands.

The connection between the speech processor and the stimulator is usually established by means of a radio frequency (RF-) link, which may utilize coils for the transcutaneous transmission of RF/power signals. Note that via the RF-link both stimulation energy and stimulation information are conveyed. Typically, digital data transfer protocols employing bit rates of some hundreds of kBit/s are used.

A totally implantable cochlear implant (TICI) is a cochlear implant system without permanently used external components such as an external speech processor. The implantable TICI typically includes a microphone and subsequent stages perform audio signal processing for the implementation of a particular stimulation strategy (e.g., CIS). It also includes stimulation electrodes, power management electronics, and a coil for the transcutaneous transmission of RF signals.

Unlike a pacemaker implant, the power supply of a TICI generally cannot be established by means of a non-rechargeable battery. This is because the overall pulse repetition rate of a cochlear implant is much higher. For example, typically about 20 kpulses/s are generated by a cochlear implant using CIS, as compared to about 1 pulse/s in a pacemaker. Besides, a cochlear implant typically performs complex audio signal processing, as compared to simple sensing tasks performed in a pacemaker. Consequently, a rechargeable battery is typically required in a cochlear implant, which needs recharging after a particular time period of operation. To recharge the battery of a TICI, an External Power Supply Module (EPSM) is used for transcutaneous transmission of RF/power signals.

As shown in FIG. 2, the typical EPSM 201 may include includes a first magnet 205, a rechargeable battery 207, and a coil 209 for transcutaneous transmission of a power signal. A TICI 202 located under the skin 203 and embedded in bone 204 typically includes a second magnet 206, a coil 208 for receiving the power signal, and a rechargeable battery 211 that is recharged with the received power signal. The first magnet 205 is positioned over the second magnet 206 such that the EPSM 201 is held against the implant 202 in an optimum position. By maintaining such a position, the external coil 209 associated with EPSM 201 can, via inductive coupling, transmit power to the coil 208 of implant 202.

To recharge the battery of the EPSM 201, the EPSM 201 typically must be opened to remove the rechargeable battery 207. The rechargeable battery 207 is then placed in a battery recharger. This process may be inconvenient and/or confusing, especially for younger children. Additionally, there is the risk that the battery 207 may be misplaced or otherwise separated from the TICI. Requiring that the EPSM 201 be opened multiple times for battery removal also complicates efforts to make the EPSM 201 waterproof.

### SUMMARY OF THE INVENTION

In accordance with an embodiment of the invention, a power supply system for an implant includes an external power supply module. The external power supply module includes a first power signal transmission module having a first coil for transmitting a first electrical power signal across the skin of a user to the implant, and a first rechargeable battery for supplying power to the power signal transmission module. The power supply system further includes an external charger for recharging the first rechargeable battery. The charger includes a second power signal transmission module having a second coil for transmitting a second electrical power signal to the first coil.

In accordance with related embodiments of the invention, the power supply system may further include the implant, the implant including a third coil for receiving the first electrical power signal. The implant may further include a second rechargeable battery for supplying power to the implant, and wherein the second rechargeable battery is recharged within the implant using the first electrical power signal. The implant may be a cochlear implant that includes, for example, a speech processor. The external power supply module may include a speech processor, wherein the speech processor transmits data from the first coil to the third coil. The external power supply may include a waterproof housing. The first power signal transmission module may include a class D switching module.

In accordance with another embodiment of the invention, a charging apparatus for recharging an external power supply module for an implant is provided. The external power supply module includes a first coil for transmitting a first electrical power signal across the skin of a user to the implant. The charging apparatus includes a second coil for transmitting a second electrical power signal to the first coil.

In accordance with another embodiment of the invention, a method of recharging a battery of an external power supply module is presented. The external power supply module includes a first coil for transmitting a first electrical power signal across the skin of a user to the implant. The external power supply module further includes a first rechargeable battery for supplying power to the power signal transmission module. The method includes transmitting a second electrical power signal from a second coil to the first coil. The first rechargeable battery is recharged with the second electrical power signal.

In accordance with related embodiments of the invention, an implant may include a third coil and a second rechargeable battery, the second rechargeable battery for supplying power to the implant. The method further includes receiving the first electrical power signal at the third coil. The second rechargeable battery is recharged with the first electrical power signal.

In further related embodiments of the invention, the implant may be a cochlear implant. The cochlear implant may include a speech processor for providing stimulation data, the method further comprising transmitting the stimulation data from the first coil to the third coil. The cochlear implant may include an array of electrodes, the method further comprising stimulating one or more of the electrodes based, at least in part, on the stimulation data.

In still further related embodiments of the invention, the method may include enclosing the external power supply module in a waterproof housing. Transmitting the second electrical power signal and recharging the first rechargeable battery may be performed without opening the waterproof housing. Transmitting the second electrical power signal may includes positioning the second coil so that it is aligned with the first coil. The external power supply module may include a class D switching module, wherein the method includes using the class D switching module to form the first electrical power signal.

### BRIEF DESCRIPTION OF THE DRAWINGS

The foregoing features of the invention will be more readily understood by reference to the following detailed description, taken with reference to the accompanying drawings, in which:

FIG. 1 illustrates a sectional view of an ear connected to a cochlear implant system (PRIOR ART);

FIG. 2 is a graphical illustration of a Totally Implantable Cochlear Implant (TICI) and external part used for transfer of power (PRIOR ART);

FIG. 3 is a graphical illustration of an external power supply module and an external charger, in accordance with an embodiment of the invention;

FIG. 4 shows an illustrative circuit of an external power supply module (EPSM) and an associated implant, in accordance to an embodiment of the present invention; and

FIG. 5 shows an illustrative circuit of an external power supply module (EPSM) including a rechargeable battery that includes a Greinacher circuit, in accordance to an embodiment of the present invention.

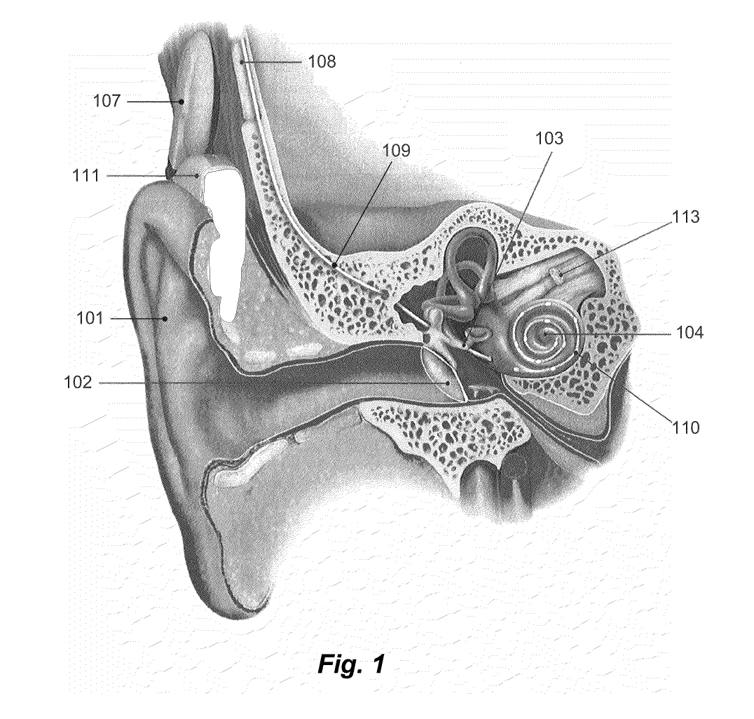

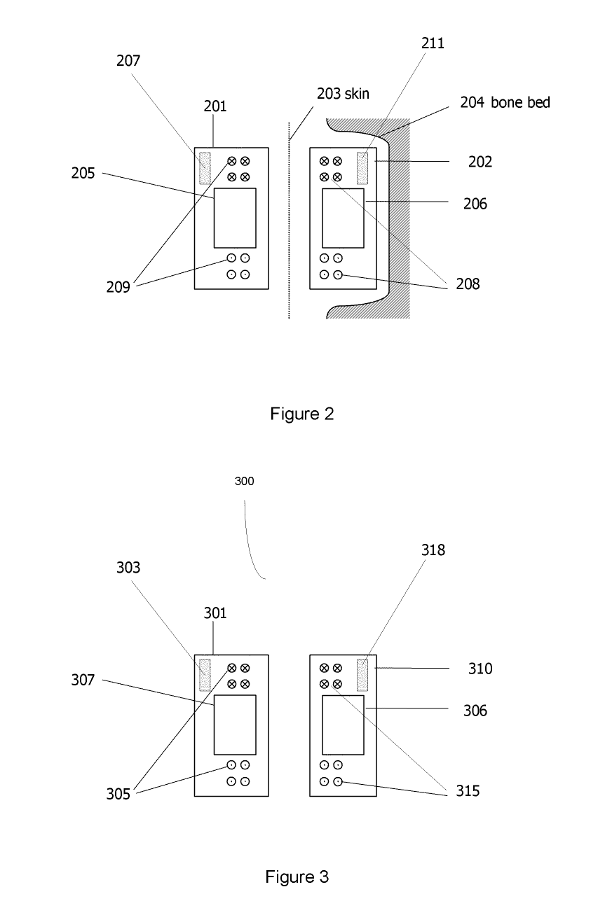

### DETAILED DESCRIPTION OF SPECIFIC EMBODIMENTS

In illustrative embodiments, a system and method for recharging a battery of an external power supply module (EPSM) associated with an implant is presented. The EPSM includes a first coil for transmitting power across the skin of a user. An external charger has a second coil that transfers power to the first coil of the EPSM, without having to open the EPSM. Various embodiments of the EPSM are advantageously made waterproof. Further details are described below.

FIG. 3 is a graphical illustration of a power system 300 that includes an EPSM 301 and an external charger 310, in accordance with an embodiment of the invention. The EPSM module 301 includes a first power signal transmission module 303. The first power signal transmission module includes a first rechargeable battery 303 and a first coil 305 for transcutaneously transmitting power from the battery 303 to any one of a variety of implants (not shown), such as a cochlear implant, a defibrillator, a cardioverter, a pacemaker, and a retinal implant. The cochlear implant may be a TICI that includes a speech processor. Alternatively, the speech processor may be located externally, such as in combination with the EPSM. In such embodiments, both power and data may be transmitted from the first coil 305 to the implant.

In various embodiments, the EPSM 301 may include a first magnet 307 that is positioned over a second magnet of the implant, such that the EPSM 301 is held against the implant in an optimum position, as described above with regard to a TICI. By maintaining such a position, the first coil 305 associated with the EPSM 301 can, via inductive coupling, transmit power to, without limitation, a third coil associated with the implant. More particularly, the first coil 305 generates a field that is picked up and converted into electric current by the third coil within the implant when it is aligned with said first coil 305. The power received by the implant may be used, for example, to provide power to electronic circuitry within the implant and/or to recharge a battery within the implant.

The external charger 310 is advantageously capable of recharging the first battery 303 without opening the EPSM 301. For example, the charger 310 may include a second power signal transmission module having a second coil 315 for transmitting a second electrical power signal to the first coil 305. By placing the EPSM 301 in an optimum position with regard to the charger 310, the charger 310 can, via inductive coupling, transmit power from the second coil 315 to the first coil 305 of the EPSM 301.

To recharge the battery of the EPSM 301, the user may place the EPSM 301 into a docking base of the charger 310. In various embodiments, the housings of the charger 310 and the EPSM 301 may be shaped such that the EPSM 301 is guided into the optimum position for transmission of power. The charger may also include a magnet 306 that works with magnet 307 of the EPSM 301 to optimally position the EPSM 301. Typically, the best performance is obtained when the two coils 305 and 315 of the EPSM 310 and charger 310, respectively, are perfectly aligned. The housing of the EPSM 301 may include, for example, teeth that mate with corresponding slots in the base of charger 310 to align the two housing portions, or various guide rails.

Various feedback may be provided to the user when the EPSM 301 is positioned in the optimum position relative to the charger 310. For example, the EPSM 301 or charger 310 may provide, without limitation, an indicator light and/or clicking sound when in the optimum position. Indications of correct operation, power transfer, voltage/current levels, and other status may also be provided in the charger 310 and/or EPSM 301.

The charger 310 may be powered by connecting to wall socket and using conventional house current. In other embodiments, the charger 310 may include its own internal battery 318, which may be rechargeable by, without limitation, plugging into an available power source. Using rechargeable batteries within the charger 310 may be convenient when a power source is not available, for example, when in an outside environment, such as camping.

In illustrative embodiments, the EPSM 301 includes a waterproof housing. This capability is more easily incorporated into the EPSM 301 of the current invention, as the user does not have open the EPSM 301 to recharge the battery 303. The housing of the EPSM 301 may be held together by, without limitation, a sealant or a weld seam. For example, ultrasonic welding or a UV curable sealant may be used.

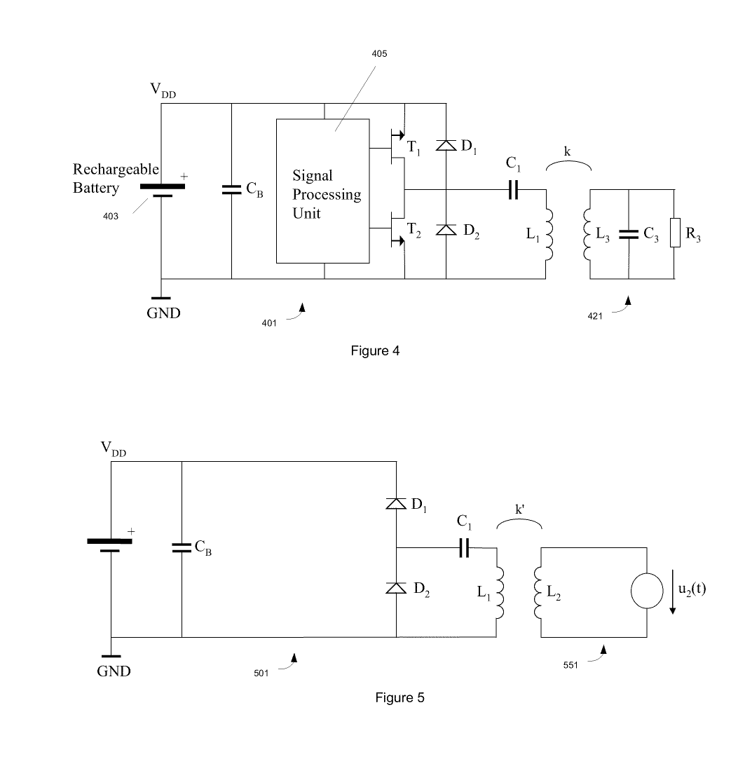

FIG. 4 shows an illustrative circuit of an EPSM 401 and an associated implant 421, in accordance to an embodiment of the present invention. The EPSM 401 advantageously includes a class-D switching scheme for transmitting, without limitation, power or data, that can be integrated onto a single microchip. Thus, power consumption and system size may be kept very small. Also, a signal processing unit 405 for driving switches T1 and T2 can easily be integrated onto such a microchip. More particularly, embodiment of the EPSM 401 may include a rechargeable battery 403, a stabilization capacitor CB, a signal processing unit 405, class-D switching transistors T1 and T2, protection diodes D1 and D2, and a series tuned RF-circuit C1, L1. The EPSM is inductively coupled to an implant (coupling factor k) 421, which is here illustratively represented by a parallel tuned RF-circuit L3, C3, and R3.

FIG. 5 shows an illustrative circuit of an EPSM 501 including a rechargeable battery 503 along with an external charger 551, in accordance to an embodiment of the present invention. The external charger 551 is illustratively represented by coil L2 driven by the RF-voltage source u2(t). Coils L2 and EPSM-coil L1 are inductively coupled (coupling factor k′). Network C1, D1, D2, CB represent the basic topology of a so called “Greinacher circuit”. The Greinacher circuit is a textbook approach for voltage doubling.

Although various exemplary embodiments of the invention have been disclosed, it should be apparent to those skilled in the art that various changes and modifications can be made that will achieve some of the advantages of the invention without departing from the true scope of the invention. These and other obvious modifications are intended to be covered by the appended claims.

What is claimed is:

1. A power supply system for an implant, the system comprising:
an external power supply module including:
a first power signal transmission module including a first coil for transmitting a first electrical power signal across the skin of a user to the implant; and
a first rechargeable battery for supplying power to the power signal transmission module; and
an external charger for recharging the first rechargeable battery; the charger including a second power signal transmission module including a second coil for transmitting a second electrical power signal to the first coil.

2. The power supply system according to claim 1, further comprising the implant, the implant including a third coil for receiving the first electrical power signal.

3. The power supply system according to claim 2, wherein the implant further includes a second rechargeable battery for supplying power to the implant, and wherein the second rechargeable battery is recharged within the implant using the first electrical power signal.

4. The power supply system according to claim 2, wherein the implant is a cochlear implant.

5. The power supply system according to claim 4, wherein the cochlear implant includes a speech processor.

6. The power supply system according to claim 4, wherein the external power supply module includes a speech processor, and wherein the speech processor transmits data from the first coil to the third coil.

7. The power supply system according to claim 1, wherein the external power supply module is enclosed in a waterproof housing.

8. The power supply system according to claim 1, wherein the charger is adapted to transmit the second electrical power signal from the second coil to the first coil without opening the waterproof housing.

9. The power supply system according to claim 1, wherein the first power signal transmission module includes a class D switching module.

10. A charging apparatus for recharging an external power supply module for an implant, the external power supply module including a first coil for transmitting a first electrical power signal across the skin of a user to the implant, the charging apparatus including:
a second coil for transmitting a second electrical power signal to the first coil.

11. The charging apparatus according to claim 10, wherein the external power supply module is enclosed in a housing, and wherein the charger is adapted to transmit the second electrical power signal from the second coil to the first coil without opening the housing.

12. A method of transferring power in an implant system, the system including an external power supply module, the external power supply module including a first coil for transmitting a first electrical power signal across the skin of a user to an implant, the external power supply module further including a first rechargeable battery for supplying power to the power signal transmission module, the method comprising:
transmitting a second electrical power signal from a second coil to the first coil; and
recharging the first rechargeable battery with the second electrical power signal.

13. The method according to claim 12, further comprising an implant, the implant including a third coil and a second rechargeable battery, the second rechargeable battery for supplying power to the implant, the method further comprising:
receiving the first electrical power signal at the third coil;
recharging the second rechargeable battery with the first electrical power signal.

14. The method according to claim 13, wherein the implant is a cochlear implant.

15. The method according to claim 13, wherein the cochlear implant includes a speech processor.

16. The method according to claim 12, wherein the external power supply module includes a speech processor for providing stimulation data, the method further comprising transmitting the stimulation data from the first coil to the third coil.

17. The method according to claim 16, wherein the cochlear implant includes an array of electrodes, the method further comprising stimulating one or more of the electrodes based, at least in part, on the stimulation data.

18. The method according to claim 12, further comprising enclosing the external power supply module in a waterproof housing.

19. The method according to claim 12, wherein transmitting the second electrical power signal and recharging the first rechargeable battery is performed without opening the waterproof housing.

20. The method according to claim 12, wherein transmitting the second electrical power signal includes positioning the second coil so that it is aligned with the first coil.

21. The method according to claim 12, wherein the external power supply module includes a class D switching module, and wherein the method includes using the class D switching module to form the first electrical power signal.
